(19) United States
(12) Patent Application Publication
(10) Pub. No.: US 2023/0299476 A1
(43) Pub. Date: Sep. 21, 2023

(54) METHOD OF FOCUSING ELECTROMAGNETIC WAVE WITH ADJUSTABLE DEPTH OF FOCUS AND OPERATION APPARATUS THEREOF

(71) Applicant: Electronica and Telecommunications Research Institute, Daejeon (KR)

(72) Inventors: Kwang Jae LEE, Daejeon (KR); Won Young SONG, Daejeon (KR); Soon Ik JEON, Daejeon (KR)

(73) Assignee: Electronics and Telecommunications Research Institute, Daejeon (KR)

(21) Appl. No.: 17/981,630

(22) Filed: Nov. 7, 2022

(30) Foreign Application Priority Data
Mar. 21, 2022 (KR) 10-2022-0034556

(51) Int. Cl. H01Q 3/24; A61B 18/18

(52) U.S. Cl. CPC H01Q 3/245; A61B 18/18; A61B 2018/00005

(57) ABSTRACT

A method of focusing radio waves, which is performed by a processor, may comprise: generating an anatomic numerical model for electromagnetic analysis inside a living body including a focusing target; calculating a current distribution, in which radio waves are focusable at a target depth inside the living body, based on the anatomic numerical model; and extracting a pattern combination of antenna modules in which the calculated current distribution is implementable, wherein the pattern combination is formed by controlling one or more antenna elements configured to radiate radio waves through switches individually coupled to two or more antenna elements.

### CROSS-REFERENCE TO RELATED APPLICATIONS

This application claims priority to Korean Patent Application No. 10-2022-0034556 filed on Mar. 21, 2022 with the Korean Intellectual Property Office (KIPO), the entire contents of which are hereby incorporated by reference.

### BACKGROUND

### 1. Technical Field

Example embodiments of the present disclosure relate to technology for focusing radio waves, and more particularly, to an apparatus for focusing radio waves with an adjustable focusing depth, which is capable of focusing radio waves on a focusing target positioned inside an object, and a method of operating the same.

### 2. Related Art

The information disclosed in this section is only to provide background information about the present example embodiments and does not form the related art.

Due to an advantage that radio wave energy has appropriate penetrating power with respect to a living body and is freely adjusted with simple equipment and radiated onto the living body, radio wave energy can be widely used for non-invasive energy treatment and power supply for a human body-implantable device or the like.

Radio wave energy is a non-invasive energy mechanism applicable to a living body, is relatively harmless as compared with radiation, and has no limitation in being applied to a structure of a bone or air and therefore radio wave energy is evaluated as excellent energy for non-invasive energy application technology.

Meanwhile, since energy is radiated into a living body without incision, non-invasive energy focusing technology can be applied to various areas, and radio wave energy should be precisely focused at various target depths. However, due to different complex radio wave characteristic environments according to application areas to which radio waves are radiated, it is difficult to provide a focusing apparatus suitable for various situations.

In particular, in the field of implantable medical devices (IMDs), due to a problem of human safety from unnecessary electromagnetic waves and limitations on a size and environment due to the characteristics of the device implanted in a human body, wireless power transmission technology requires a design with which power can be supplied limitedly only to a target area. However, when a focusing depth cannot be adjusted according to various areas into which the IMD is inserted or a depth to which the IMD is inserted, it is difficult for the IMD to exhibit desired performance.

### SUMMARY

Accordingly, example embodiments of the present disclosure are provided to substantially obviate one or more problems due to limitations and disadvantages of the related art.

Example embodiments of the present disclosure provide a method and apparatus for focusing radio waves which is capable of adjusting a focusing depth of radio waves according to a depth of a focusing target positioned inside an object such as a human body.

According to a first exemplary embodiment of the present disclosure, a method of focusing radio waves, which is performed by a processor, may comprise: generating an anatomic numerical model for electromagnetic analysis inside a living body including a focusing target; calculating a current distribution, in which radio waves are focusable at a target depth inside the living body, based on the anatomic numerical model; and extracting a pattern combination of antenna modules in which the calculated current distribution is implementable, wherein the pattern combination is formed by controlling one or more antenna elements configured to radiate radio waves through switches individually coupled to two or more antenna elements.

The generating of the anatomic numerical model may further include measuring radio wave characteristic information including permittivity, conductivity, and/or impedance through a radio wave characteristic measurement module.

The generating of the anatomic numerical model may include acquiring external information that includes a target area and a target size of the living body including the focusing target.

The pattern combination may be extracted by combining channel weights according to an arrangement of antenna element pattern modules.

In the antenna element pattern modules, the two or more antenna elements may be formed in concentric circle shapes having different radii.

In the antenna element pattern modules, two or more antenna elements may be disposed in a grid form on a plane.

According to a second exemplary embodiment of the present disclosure, an apparatus for focusing radio waves with an adjustable focusing depth may comprise: a memory in which one or more instructions are stored; and a processor configured to execute the one or more instructions stored in the memory, wherein: the processor configured to execute the one or more instructions performs an operation of generating an anatomic numerical model for electromagnetic analysis inside a living body including a focusing target, calculating a current distribution, in which radio waves are focusable at a target depth inside the living body, based on the anatomic numerical model, and an operation of extracting a pattern combination of antenna modules in which the calculated current distribution is implementable; and the pattern combination is formed by controlling one or more antenna elements configured to radiate radio waves through switches individually coupled to two or more antenna elements.

The operation of, by the processor, generating the anatomic numerical model may further include measuring radio wave characteristic information including permittivity, conductivity, and/or impedance through a radio wave characteristic measurement module.

The operation of, by the processor, generating the anatomic numerical model may further include acquiring external information that includes a target area and a target size of the living body including the focusing target.

The pattern combination may be extracted by combining channel weights according to an arrangement of antenna element pattern modules.

In the antenna element pattern modules, two or more antenna elements may be formed in concentric circle shapes having different radii.

In the antenna element pattern modules, the two or more antenna elements may be disposed in a grid form on a plane.

According to a third exemplary embodiment of the present disclosure, an apparatus for focusing radio waves with an adjustable focusing depth may comprise: a radio wave focusing adjustment module; and a focusing antenna module, wherein: the radio wave focusing adjustment module includes an element pattern forming module configured to adjust an antenna pattern, a high-power radio wave energy generating module configured to adjust a parameter of an antenna element, and a calculation module configured to calculate parameters of the antenna pattern and the antenna element based on electromagnetic characteristic information inside an object and characteristic information of radio waves to be radiated; and the focusing antenna module further includes the antenna element configured to radiate the radio waves onto a focusing target, and a switch configured to connect the element pattern forming module and the antenna element and adjust power of the antenna element.

The radio wave focusing adjustment module may further include a radio wave measurement module configured to measure radio wave information inside the object.

The antenna module may include two or more antenna elements.

The antenna elements may be disposed in concentric circle shapes having different radii to form one antenna element pattern module.

The antenna module may include two or more antenna element pattern modules.

In the antenna module, the two or more antenna elements may be disposed in a grid form on a plane.

The focusing antenna module may further include an interface configured to maintain a constant distance between the focusing antenna module and the focusing target.

The interface may include a matching medium through which radio waves are matched and transmitted to a living body and which cools surface tissue of the object.

According to the present disclosure, it possible to precisely and efficiently focus radio wave energy at various target depths of an object, and a radio wave characteristic environment of a target area is reflected on a device in the form of information, thereby applying the present disclosure to devices implanted at various depths in various areas of the object.

According to the present disclosure, it possible to precisely and efficiently focus radio wave energy at various depths in various areas of an object, thereby providing technology for applying radio wave energy to focusing targets, that is, lesions, positioned at various depths of a complex body area in the field of non-invasive energy treatment technology.

In addition, in the field of wireless power supply for a human body-implantable device, it is possible to provide technology for securing human safety from unnecessary electromagnetic waves through focusing of radio wave energy and also supplying required power according to various areas or insertion depths.

### BRIEF DESCRIPTION OF DRAWINGS

FIG. 1 is a block diagram of an apparatus for focusing radio waves with an adjustable focusing depth according to one example embodiment of the present disclosure.

FIGS. 2A and 2B are block diagrams of an antenna module according to one example embodiment of the present disclosure.

FIGS. 3A to 3C show diagrams illustrating antenna elements being individually controlled according to one example embodiment of the present disclosure.

FIGS. 4A to 4C show diagrams illustrating a method of adjusting a focusing depth according to one example embodiment of the present disclosure.

FIG. 5 is a diagram illustrating an arrangement of a plurality of antenna element pattern modules according to one example embodiment of the present disclosure.

FIGS. 6A to 6C show diagrams illustrating an antenna module 110 and pattern formation according to another example embodiment of the present disclosure.

FIG. 7 is a flowchart of a method of focusing radio waves with an adjustable focusing depth according to one example embodiment of the present disclosure.

### DETAILED DESCRIPTION OF THE EMBODIMENTS

Embodiments of the present disclosure are disclosed herein. However, specific structural and functional details disclosed herein are merely representative for purposes of describing embodiments of the present disclosure. Thus, embodiments of the present disclosure may be embodied in many alternate forms and should not be construed as limited to embodiments of the present disclosure set forth herein.

Accordingly, while the present disclosure is capable of various modifications and alternative forms, specific embodiments thereof are shown by way of example in the drawings and will herein be described in detail. It should be understood, however, that there is no intent to limit the present disclosure to the particular forms disclosed, but on the contrary, the present disclosure is to cover all modifications, equivalents, and alternatives falling within the spirit and scope of the present disclosure. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

In exemplary embodiments of the present disclosure, “at least one of A and B” may refer to “at least one of A or B” or “at least one of combinations of one or more of A and B”. In addition, “one or more of A and B” may refer to “one or more of A or B” or “one or more of combinations of one or more of A and B”.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between,” “adjacent” versus “directly adjacent,” etc.).

The terminology used herein is for the purpose of describing particular embodiments only and is not intended to be limiting of the present disclosure. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this present disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, preferred exemplary embodiments of the present disclosure will be described in more detail with reference to the accompanying drawings. In describing the present disclosure, in order to facilitate an overall understanding, the same reference numerals are used for the same elements in the drawings, and duplicate descriptions for the same elements are omitted.

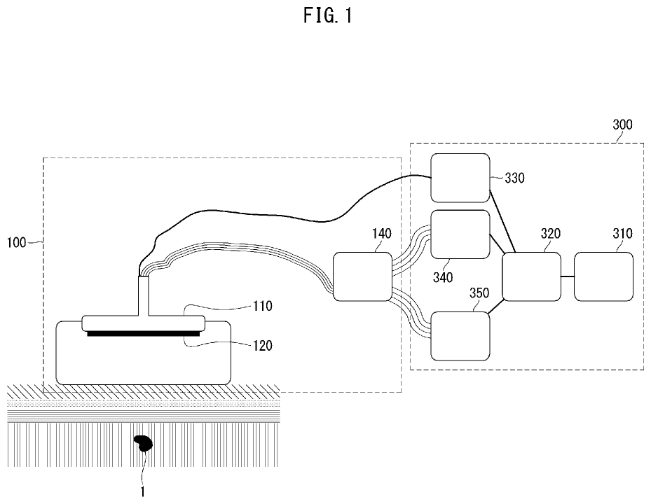

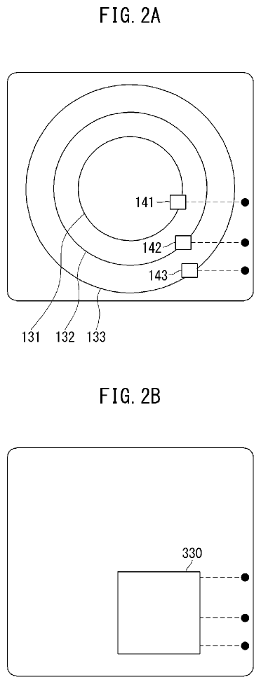

FIG. 1 is a block diagram of an apparatus for focusing radio waves with an adjustable focusing depth according to one example embodiment of the present disclosure. FIGS. 2A and 2B are block diagrams of an antenna module according to one example embodiment of the present disclosure.

The apparatus for focusing radio waves with an adjustable focus depth includes a focusing antenna module 100 for radiating radio waves onto a focusing target 1 and a radio wave focusing adjustment module 300 for controlling the focusing antenna module 100.

Referring to FIGS. 1, 2A and 2B, the focusing antenna module 100 may include an antenna element 130, a switch 140, and an interface 150. The antenna element 130 may be connected to an antenna element pattern forming module 330 through the switch 140. The antenna element 130 may generate radio waves according to whether the switch 140 is on or off. A plurality of antenna elements 130 may be provided. One or more antenna elements 130 (131, 132, and 133) may constitute an antenna element pattern module 120. The one or more antenna elements 130 (131, 132, and 133) constituting the antenna element pattern module 120 may all be coupled to the antenna element pattern forming module 330 through individually coupled switches.

The switch 140 may connect the antenna element pattern forming module 330 and the antenna element 130. The switch 140 may turn the antenna element 130 on/off under the control of the antenna element pattern forming module 330 to adjust the generation of radio waves. As long as the switch 140 is technology capable of controlling the operation of the antenna element 130 under the control of the antenna element pattern forming module 330, various switches are applicable.

The interface 150 may maintain a constant distance between the antenna element and the focusing target 1. The interface 150 may be filled with a matching medium that allows the radio waves radiated through the focusing antenna module 100 to be matched and transmitted to a living body. Here, the matching medium may serve to prevent overheating generated on a surface of biological tissue.

Referring again to FIG. 1, the radio wave focusing adjustment module 300 may further include a calculation module 310, a system control module 320, an element pattern forming module 330, a high-power radio wave energy generating module 340, and a radio wave measurement module 350.

The radio wave measurement module 350 may acquire radio wave characteristic information of an object by radiating radio waves to be radiated. Here, the radio wave characteristic information may be permittivity, conductivity, impedance, or the like which is effectively determined by a structure of complex biological tissue such as bone, muscle, or fat. Also, the radio wave measurement module 350 may receive external information. Here, the external information may be information about an area or the like in which the focusing target 1 is positioned. In particular, in the case of a human body, the external information may be information about an application area such as a chest or an abdomen or information about a body shape of an object.

The calculation module 310 may generate an anatomic numerical model of the object based on the radio wave characteristic information and/or the external information. The anatomic numerical model of the object may be modeled to enable electromagnetic analysis inside the object, and an antenna element pattern may be calculated to have a focusing depth of a target point based on the anatomic numerical model. The calculation module 310 may derive an optimal antenna current distribution from various antenna current distributions according to a depth of the focusing target 1. The calculation module 310 may determine the antenna current distribution based on permittivity, conductivity, impedance, and a target depth of the anatomic numerical model. The calculation module 310 may extract an antenna element pattern capable of forming the calculated optimal antenna current distribution.

The antenna element pattern forming module 330 may implement the antenna element pattern derived through the calculation module 310. The antenna element pattern module may form a specific antenna element pattern by adjusting a switch connected to the antenna element. The antenna element pattern forming module 330 may shorten a time required for forming an element pattern according to a target depth value through information about a pattern combination in which various antenna element patterns are formed according to a target depth.

The high-power radio wave energy generating module 340 may generate high-power radio wave energy in the antenna element pattern formed by the antenna element pattern forming module 330 controlling the switch. The high-power radio wave energy generating module 340 may generate radio wave energy to apply high-power radio wave energy to a point of the focusing target 1.

The system control module 320 may control the operations of the calculation module 310, the element pattern forming module 330, the high-power radio wave energy generating module 340, and the radio wave measurement module 350.

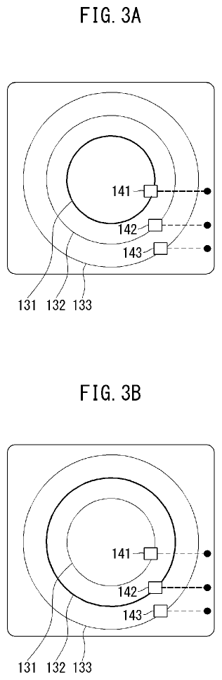

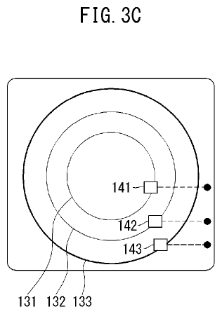

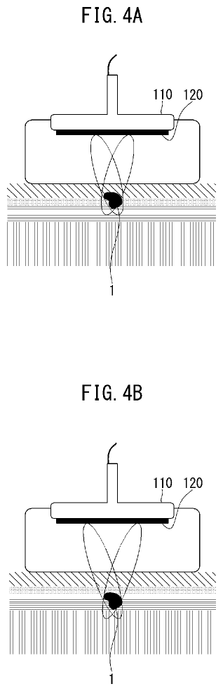

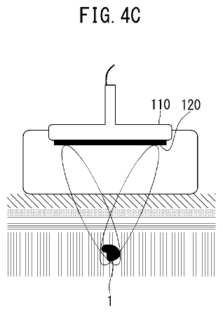

FIGS. 3A to 3C show diagrams illustrating antenna elements being individually controlled according to one example embodiment of the present disclosure. FIGS. 4A to 4C show diagrams illustrating a method of adjusting a focusing depth according to one example embodiment of the present disclosure.

Referring to FIGS. 3A to 3C, an antenna module 110 including three antenna elements is shown. In the antenna module 110, the three antenna elements are connected through individual switches. Referring to FIG. 3A, the element pattern forming module 330 may activate only a first antenna element 131 when only a first switch 141 is operated to be turned on and a second switch 142 and a third switch 143 are adjusted to be turned off. Referring to FIG. 3B, the element pattern forming module 330 may activate only the second antenna element 132 when only the second switch 142 is operated to be turned on and the first switch 141 and the third switch 143 are adjusted to be turned off. Referring to FIG. 3C, the element pattern forming module 330 may activate only the third antenna element 133 when only the third switch 143 is operated to be turned on and the first switch 141 and the second switch 142 are adjusted to be turned off. Here, referring to FIGS. 4A to 4C, when the third antenna element 133 is activated, a deeper focusing depth may be implemented as compared with a case in which the first antenna element 131 is activated. In addition, when the second antenna element 132 is activated, a medium focusing depth may be implemented. FIGS. 3A to 3C and 4A to 4C exemplarily show that only one antenna element 131, 132, or 133 is activated, but the present disclosure is not limited thereto. Various methods may be implemented, such as concurrently activating the first antenna element 131 and the third antenna element 133 or concurrently activating the second antenna element 132 and the third antenna element 133. In addition, the number of antenna elements 130 is not limited to three, more antenna elements 130 may be provided, and a focusing depth may be adjusted through various combinations in which two or more antenna elements 130 are activated according to the number of antenna elements 130.

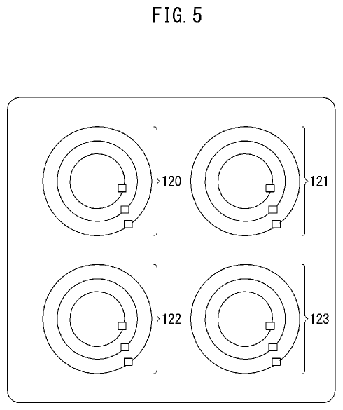

FIG. 5 is a diagram illustrating an arrangement of a plurality of antenna element pattern modules according to one example embodiment of the present disclosure.

The antenna module 110 may include at least one antenna element pattern module 120. By adjusting a plurality of antenna element pattern modules 121, 122, and 123 in addition to adjusting a focusing depth according to a combination of the individual antenna elements 130, radio waves may be radiated at a wider variety of focusing depths. In addition, by adjusting weights according to the antenna element pattern modules 120, 121, 122, and 123, a magnitude and a phase of radiated power may also be adjusted. In particular, the element pattern forming module 330 may determine various pattern combinations by adjusting weights of the antenna element 130 and the antenna element pattern module 120.

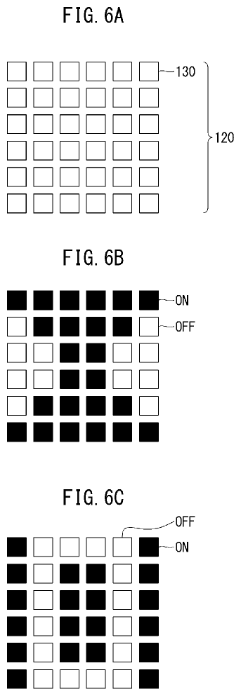

FIGS. 6A to 6C show diagrams illustrating an antenna module 110 and pattern formation according to another example embodiment of the present disclosure.

Referring to FIG. 6A, the antenna module 110 may have a structure in which a plurality of antenna elements 130 are combined in a grid form on a radial plane. As in the case of FIGS. 2 to 5, the antenna elements 130 may include individual switches. The antenna elements 130 may be connected to the antenna element pattern forming module 330 through the switches 140 that are individually coupled.

Referring to FIGS. 6B and 6C, the antenna module 110 may generate various antenna element patterns by adjusting whether the switch 140 coupled to the antenna element 130 is on or off.

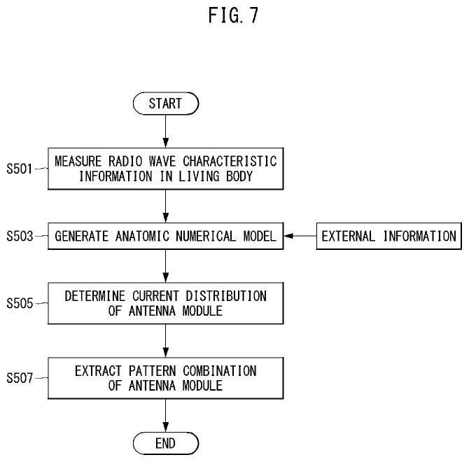

FIG. 7 is a flowchart of a method of focusing radio waves with an adjustable focusing depth according to one example embodiment of the present disclosure.

First, radio wave characteristic information of an object including a focusing target 1 may be measured and obtained through the radio wave measurement module 350 (S501). Here, the radio wave characteristic information may be permittivity, conductivity, impedance, or the like which is effectively determined by a structure of complex biological tissue such as bone, muscle, or fat. The calculation module 310 may generate an anatomic numerical model modeled to enable electromagnetic analysis of the object including the focusing target 1 based on the radio wave specific information of the object (S503). In this case, the calculation module 310 may additionally consider received external information in generating the anatomic numerical model. Here, the external information may be information about an area in which the focusing target 1 is positioned. In particular, when the object is a human body, the external information may be information about an application area such as a chest or an abdomen or information of a body shape. A Green function for electromagnetic analysis inside a living body may be obtained through the anatomic numerical model. The calculation module 310 may determine a current distribution of an antenna element based on the anatomic numerical model and a focusing depth of the focusing target 1 (S505). In operation S505, an optimal antenna current distribution may be calculated according to permittivity, conductivity, impedance, a target depth, or the like of the anatomic numerical model. The antenna element pattern forming module 330 may combine antenna element patterns according to the current distribution of the antenna element determined by the calculation module 310 (S507). The antenna element pattern forming module 330 may form an antenna element pattern having a target focusing depth by short-circuiting a switch according to the antenna element pattern. The antenna element pattern forming module 330 may have various pattern combinations capable of implementing a target depth and a magnitude and a phase of power at the target depth to focus radio waves at various target depths. Finally, high-power radio wave energy may be generated in the implemented antenna element pattern, thereby radiating radio wave energy onto the focusing target.

The operations of the method according to the exemplary embodiment of the present disclosure can be implemented as a computer readable program or code in a computer readable recording medium. The computer readable recording medium may include all kinds of recording apparatus for storing data which can be read by a computer system. Furthermore, the computer readable recording medium may store and execute programs or codes which can be distributed in computer systems connected through a network and read through computers in a distributed manner.

The computer readable recording medium may include a hardware apparatus which is specifically configured to store and execute a program command, such as a ROM, RAM or flash memory. The program command may include not only machine language codes created by a compiler, but also high-level language codes which can be executed by a computer using an interpreter.

Although some aspects of the present disclosure have been described in the context of the apparatus, the aspects may indicate the corresponding descriptions according to the method, and the blocks or apparatus may correspond to the steps of the method or the features of the steps. Similarly, the aspects described in the context of the method may be expressed as the features of the corresponding blocks or items or the corresponding apparatus. Some or all of the steps of the method may be executed by (or using) a hardware apparatus such as a microprocessor, a programmable computer or an electronic circuit. In some embodiments, one or more of the most important steps of the method may be executed by such an apparatus.

In some exemplary embodiments, a programmable logic device such as a field-programmable gate array may be used to perform some or all of functions of the methods described herein. In some exemplary embodiments, the field-programmable gate array may be operated with a microprocessor to perform one of the methods described herein. In general, the methods are preferably performed by a certain hardware device.

The description of the disclosure is merely exemplary in nature and, thus, variations that do not depart from the substance of the disclosure are intended to be within the scope of the disclosure. Such variations are not to be regarded as a departure from the spirit and scope of the disclosure. Thus, it will be understood by those of ordinary skill in the art that various changes in form and details may be made without departing from the spirit and scope as defined by the following claims.

What is claimed is:

1. A method of focusing radio waves, which is performed by a processor, the method comprising:
generating an anatomic numerical model for electromagnetic analysis inside a living body including a focusing target;
calculating a current distribution, in which radio waves are focusable at a target depth inside the living body, based on the anatomic numerical model; and
extracting a pattern combination of antenna modules in which the calculated current distribution is implementable,
wherein the pattern combination is formed by controlling one or more antenna elements configured to radiate radio waves through switches individually coupled to two or more antenna elements.

2. The method of claim 1, wherein the generating of the anatomic numerical model further includes measuring radio wave characteristic information including permittivity, conductivity, and/or impedance through a radio wave characteristic measurement module.

3. The method of claim 1, wherein the generating of the anatomic numerical model includes acquiring external information that includes a target area and a target size of the living body including the focusing target.

4. The method of claim 1, wherein the pattern combination is extracted by combining channel weights according to an arrangement of antenna element pattern modules.

5. The method of claim 4, wherein, in the antenna element pattern modules, the two or more antenna elements are formed in concentric circle shapes having different radii.

6. The method of claim 4, wherein, in the antenna element pattern modules, two or more antenna elements are disposed in a grid form on a plane.

7. An apparatus for focusing radio waves with an adjustable focusing depth, the apparatus comprising:
a memory in which one or more instructions are stored; and
a processor configured to execute the one or more instructions stored in the memory,
wherein:
the processor configured to execute the one or more instructions performs an operation of generating an anatomic numerical model for electromagnetic analysis inside a living body including a focusing target, calculating a current distribution, in which radio waves are focusable at a target depth inside the living body, based on the anatomic numerical model, and an operation of extracting a pattern combination of antenna modules in which the calculated current distribution is implementable; and
the pattern combination is formed by controlling one or more antenna elements configured to radiate radio waves through switches individually coupled to two or more antenna elements.

8. The apparatus of claim 7, wherein the operation of, by the processor, generating the anatomic numerical model further includes measuring radio wave characteristic information including permittivity, conductivity, and/or impedance through a radio wave characteristic measurement module.

9. The apparatus of claim 7, wherein the operation of, by the processor, generating the anatomic numerical model further includes acquiring external information that includes a target area and a target size of the living body including the focusing target.

10. The apparatus of claim 7, wherein the pattern combination is extracted by combining channel weights according to an arrangement of antenna element pattern modules.

11. The apparatus of claim 10, wherein, in the antenna element pattern modules, two or more antenna elements are formed in concentric circle shapes having different radii.

12. The apparatus of claim 11, wherein, in the antenna element pattern modules, the two or more antenna elements are disposed in a grid form on a plane.

13. An apparatus for focusing radio waves with an adjustable focusing depth, the apparatus comprising:
a radio wave focusing adjustment module; and
a focusing antenna module,
wherein:
the radio wave focusing adjustment module includes an element pattern forming module configured to adjust an antenna pattern, a high-power radio wave energy generating module configured to adjust a parameter of an antenna element, and a calculation module configured to calculate parameters of the antenna pattern and the antenna element based on electromagnetic characteristic information inside an object and characteristic information of radio waves to be radiated; and
the focusing antenna module further includes the antenna element configured to radiate the radio waves onto a focusing target, and a switch configured to connect the element pattern forming module and the antenna element and adjust power of the antenna element.

14. The apparatus of claim 13, wherein the radio wave focusing adjustment module further includes a radio wave measurement module configured to measure radio wave information inside the object.

15. The apparatus of claim 13, wherein the antenna module includes two or more antenna elements.

16. The apparatus of claim 15, wherein the antenna elements are disposed in concentric circle shapes having different radii to form one antenna element pattern module.

17. The apparatus of claim 16, wherein the antenna module includes two or more antenna element pattern modules.

18. The apparatus of claim 15, wherein, in the antenna module, the two or more antenna elements are disposed in a grid form on a plane.

19. The apparatus of claim 13, wherein the focusing antenna module further includes an interface configured to maintain a constant distance between the focusing antenna module and the focusing target.

20. The apparatus of claim 19, wherein the interface includes a matching medium through which radio waves are matched and transmitted to a living body and which cools surface tissue of the object.
